(19) United States
(12) Patent Application Publication
(10) Pub. No.: US 2025/0295486 A1
(43) Pub. Date: Sep. 25, 2025

(54) HYDRAULIC ACCUMULATION IMPLANT SYSTEMS

(71) Applicant: EDWARDS LIFESCIENCES CORPORATION, Irvine, CA (US)

(72) Inventors: Yuval Kasher, Kfar Shmuel (IL); Dikla Kersh, Karkur (IL)

(73) Assignee: EDWARDS LIFESCIENCES CORPORATION, Irvine, CA (US)

(21) Appl. No.: 19/174,828

(22) Filed: Apr. 9, 2025

Related U.S. Application Data
(63) Continuation of application No. PCT/US2023/076933, filed on Oct. 15, 2023.
(60) Provisional application No. 63/379,437, filed on Oct. 13, 2022.

(51) Int. Cl. A61F 2/06

(52) U.S. Cl. CPC A61F 2/064; A61F 2250/0003

(57) ABSTRACT

A method involves percutaneously delivering an implant into a first blood vessel. The implant may have a flexible and/or elastic structure. The method also involves extending at least a distal portion of the implant out of the first blood vessel while retaining at least a proximal portion of the implant within the first blood vessel and injecting the implant with a fluid. The fluid is freely moveable between the proximal portion and the distal portion. The proximal portion may be shape-set to form a bulb.

### RELATED APPLICATION

This application is a continuation of International Patent Application No. PCT/US2023/076933, filed Oct. 15, 2023, which claims the benefit of U.S. Provisional Patent Application No. 63/379,437, filed on Oct. 13, 2022, the complete disclosures of which are hereby incorporated by reference in their entireties.

### BACKGROUND

### Field

The present disclosure generally relates to the field of medical implant devices and procedures.

### Description of Related Art

Insufficient or reduced compliance in certain blood vessels, including arteries such as the aorta, can result in reduced perfusion, cardiac output, and other health complications. Increasing compliance and/or otherwise controlling flow in such blood vessels can improve patient outcomes.

### SUMMARY

Described herein are devices, methods, and systems that facilitate the restoration and/or enhancement of compliance characteristics for target blood vessels. Devices associated with the various examples of the present disclosure can include one or more hydraulic accumulation implants configured to convey a fluid between a proximal end disposed within a blood vessel and a distal end disposed outside the blood vessel. Some example systems can include a stent configured to sandwich a hydraulic accumulation implant between the stent and an inner wall of the blood vessel.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIGS. 1A-1C illustrate example representations of cardiac and vascular anatomy of a patient.

FIGS. 2A and 2C provide cross-sectional and side views, respectively, of a blood vessel experiencing compliant expansion during the systolic phase of the cardiac cycle.

FIGS. 2B and 2D provide cross-sectional and side views, respectively, of a blood vessel experiencing compliant contraction during the systolic phase of the cardiac cycle.

FIGS. 3A and 3B provide cross-sectional and side views, respectively, a blood vessel with reduced compliance.

FIG. 4 is a graph illustrating blood pressure over time in an example healthy patient.

FIG. 5 is a graph illustrating blood pressure over time in an example patient having reduced aortic compliance.

FIGS. 6A-6D illustrate an example implant (e.g., hydraulic accumulator) configured to modulate blood pressures through one or more blood vessels of a heart in accordance with one or more examples.

FIGS. 7A and 7B illustrate an example implant (e.g., accumulator) implanted in a blood vessel (e.g., an aorta) to modulate blood pressures through the blood vessel 715 in accordance with one or more examples.

FIGS. 8A and 8B illustrate an example implant (e.g., accumulator) and stent configured to modulate blood pressures through one or more blood vessels of a heart in accordance with one or more examples.

FIGS. 9A-9D illustrate a flow diagram for a process delivering and/or implanting a compliance-enhancing system including one or more accumulators (e.g., inflatable implants) and/or stents in accordance with one or more examples.

FIGS. 10A-10D provide images of the compliance-enhancing system and certain anatomy corresponding to operations of the process of FIGS. 9A-9D according to one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, compliance-enhancement implant devices implanted in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

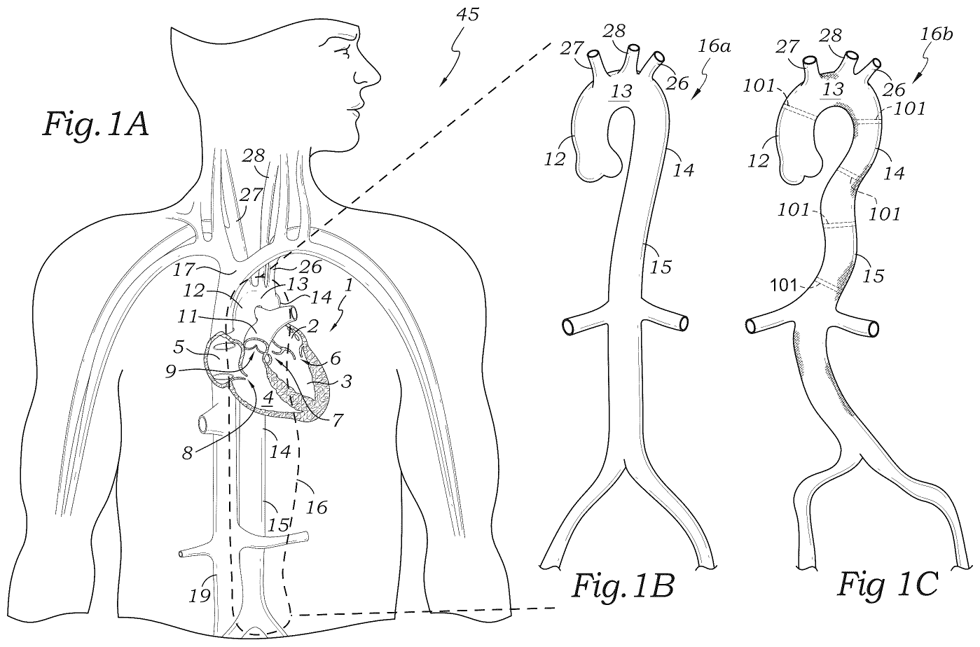

FIG. 1A illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the ascending aortic trunk 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior venae cavae, carry blood back to the heart.

FIGS. 1B and 1C show detailed views of example healthy 16a and aged/stiff 16b aortas, respectively. The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart and is sometimes referred to as the aortic ‘trunk.’ The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively frequently affected by aneurysms and dissections, often requiring open heart surgery to be repaired. The transition from the ascending aorta 12 to the aortic arch 13 is at the pericardial reflection on the aorta 16. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions (e.g., lesser volume) as transmural pressure decreases.

Arterial compliance facilitates perfusion of organs in the body with oxygenated blood from the heart. Generally, a healthy aorta and other major arteries in the body are at least partially clastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta 16a, as shown in FIG. 1B, runs along a generally straight path, whereas an aged and/or stiffened aorta 16b, as shown in FIG. 1C, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less clastic. As such conditions develop, arterial blood pressure (e.g., left-ventricular afterload) can become more pulsatile, which can have deleterious effects, such as the thickening of the left ventricle (LV) muscle, and insufficient perfusion of the heart. Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

Examples of the present disclosure provide compliance-enhancing implant devices, which may be implanted in one or more locations in a compromised aorta and/or other vessel(s). For example, FIG. 1C shows example positions of compliance-enhancing implant devices 101 implanted in various areas of an aorta 16b. Embodiments of the present disclosure provide elastic reshaping of a target blood vessel, such as the aorta, in a manner as to produce a volume differential between high- and low-pressure states, thereby mimicking conditions of a stretchy, healthy blood vessel.

Example compliance-enhancing implant devices 101 can comprise an inflatable and/or expandable accumulation device configured to be deployed at least partially within a blood vessel and/or at least partially outside the blood vessel. For example, a device 101 can comprise a proximal end deployed in the vessel and/or a distal end deployed outside the vessel. The device 101 can comprise a closed-volume chamber that can be at least partially inflated with fluid or gas during a procedure (e.g., via a dedicated port at the proximal end and/or distal end). The device 101 may be configured to prevent blood inflow as blood inflow can cause thrombosis due to turbulent blood flow and/or stagnant blood caught in the device 101.

In some examples, the device 101 can comprise a covered stent that can be deployed in parallel with the accumulation device in a manner that seals and/or creates hemostasis proximally and distally to the portion of the accumulation device that resides in the vessel (e.g., the proximal portion).

Blood may be allowed to flow through the covered stent and/or may be prevented from leaking outside the vessel through a deployment ostium of the accumulation device. The proximal portion of the accumulation device can be exposed to incident blood flow (e.g., aortic blood pressure) through a compliant wall of the covered stent. As blood pressure rises during systole, blood flow may act against the wall of the covered stent and/or drain the volume from the proximal portion of the accumulation device to its distal portion (e.g., outside the vessel).

The distal portion of the accumulation device can form a bulb (e.g., increased diameter relative to a midsection of the accumulation device) and/or may have a generally uniform size and/or shape relative to other portions of the accumulation device. In some examples, the proximal portion and/or distal portion can have some elastic resistance due to various features (e.g. rubber and/or similar material, an array of circumferential springs, and/or a cylindrical stent over a cylindrical balloon) of the accumulation device. In this way, at least a portion of the accumulation device may be configured to surrender to the systolic pressure and/or can overcome the blood pressure during diastole to drain the distal portion back to the proximal portion of the accumulation device. Thus, the accumulation device can act as a resonating compliance chamber during the cardiac cycles.

The proximal and/or distal portions of the accumulation device can have any suitable size and/or shape. In some examples, the proximal portion and/or distal portion can form a bulb having an oval and/or disc shape. In some examples, the accumulation device can comprise an elongate and/or cylindrical balloon threaded through a cylindrical stent of radial elasticity with different mechanical characteristics applied to proximal and/or distal portions of the accumulation device. The stent and/or accumulation device may be shape-set to a curved form (e.g., forming a 90-degree bend).

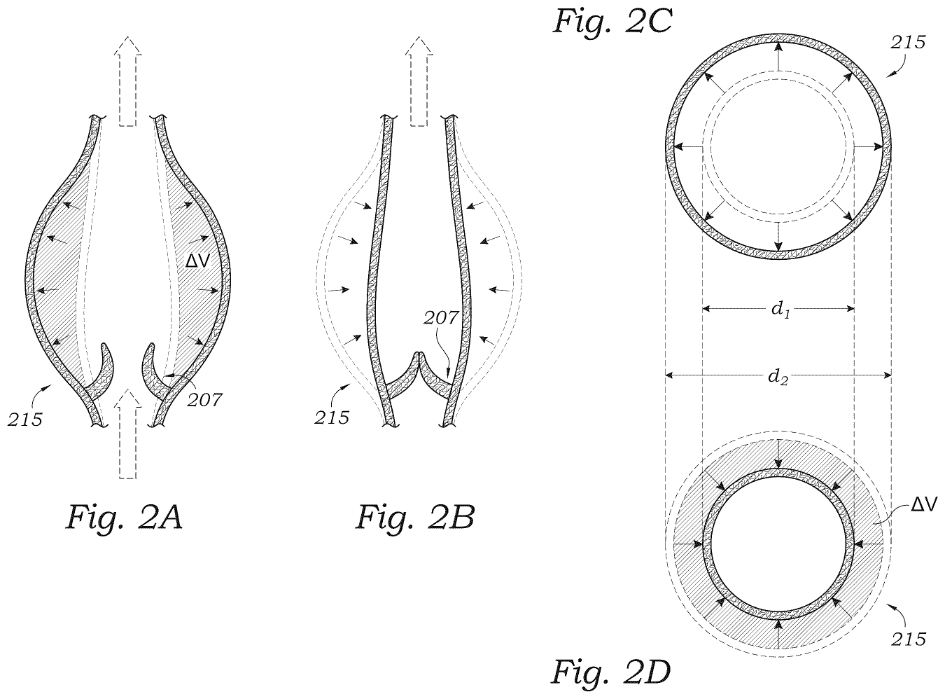

FIGS. 2A and 2C provide side and cross-sectional views, respectively, of a compliant blood vessel 215, such as an artery (e.g., aorta), experiencing expansion during the systolic phase of the cardiac cycle. FIGS. 2B and 2D provide side and cross-sectional views, respectively, of the compliant blood vessel 215 radially contracting/recoiling during the diastolic phase of the cardiac cycle. As understood by those having ordinary skill in the art, the systolic phase of the cardiac cycle is associated with the pumping phase of the left ventricle, while the diastolic phase of the cardiac cycle is associated with the filling phase of the left ventricle. As identified in FIGS. 2A and 2D, with proper arterial compliance, a change in volume 4V will generally occur in an artery between high- and low-pressure phases of the cardiac cycle. With respect to the aorta, as shown in FIGS. 2A and 2C, as blood is pumped into the aorta 215 through the aortic valve 207, the pressure in the aorta increases and the diameter of at least a portion of the aorta expands. While the blood vessel 215 is shown forming a bulge, dilation of the blood vessel 215 may involve a generally uniform expansion of the blood vessel 215 rather than a localized bulge. A first portion of the blood entering the aorta 215 during systole may pass through the aorta during the systolic phase, while a second portion (e.g., approximately half of the total blood volume) may be stored in the expanded volume 4V (see FIG. 2A) caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Arterial compliance restoration devices, methods, and concepts disclosed herein may be generally described in the context of the ascending aorta. However, it should be understood that such devices, methods and/or concepts may be applicable in connection with any other artery or blood vessel.

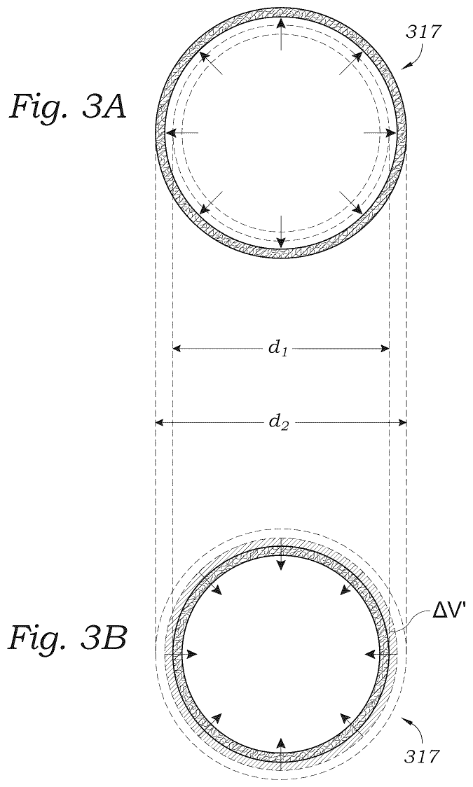

FIGS. 3A and 3B show a cross-sectional profile of a blood vessel 317 that is relatively stiff, similar to the blood vessel 16b shown in FIG. 1C, wherein the compliance of the vessel portion 317 is diminished relative to the healthy aorta 16a as shown in FIG. 1B. Due to the stiffness of the blood vessel wall, the blood vessel 317 may expand a relatively limited amount ΔV′ between diastole (shown in FIG. 3B) and systole (shown in FIG. 3A). That is, during systole, the increased fluid pressure within the blood vessel 317 may result only in a relatively small and/or negligible expansion of the diameter of the blood vessel 317, as shown with respect to the difference between the contracted diameter d₁ and the expanded diameter d₂ in FIGS. 3A and 3B. Due to the limited expansion of the blood vessel 317, the change in volume ΔV′ in the blood vessel between phases of the cardiac cycle may likewise be limited, and therefore relatively little energy is stored in the blood vessel wall in high-pressure conditions and returned to the blood circulation during low-pressure conditions, resulting in more pulsatile blood flow compared to healthy, compliant aortic tissue.

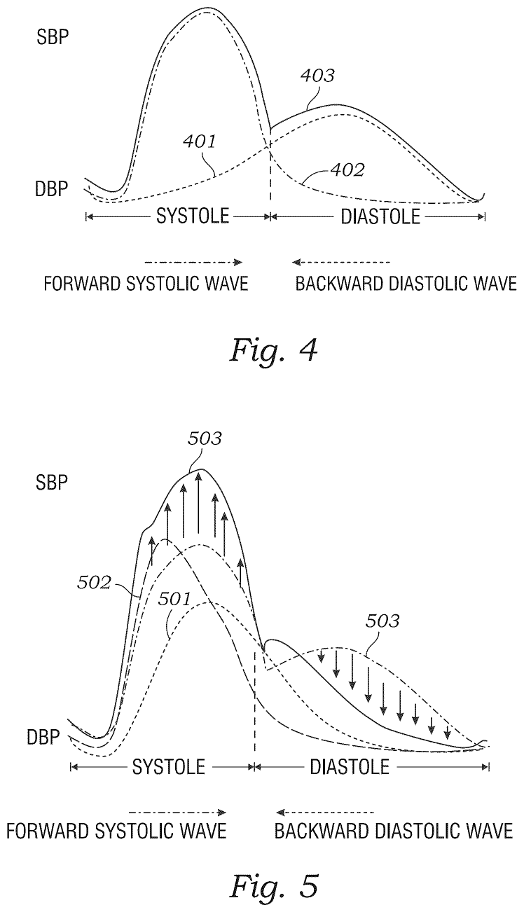

FIG. 4 is a graph illustrating blood pressure over time in an example patient with a healthy, compliant aorta, wherein arterial blood pressure is represented as a combination of a forward systolic pressure wave 402 and a backward diastolic pressure wave 401. The combination of the systolic wave 402 and the diastolic wave 401 are approximately represented by the waveform 403.

FIG. 5 is a graph illustrating blood pressure over time in an example patient having reduced aortic compliance. The graph of FIG. 5 shows, for reference purposes, the example combined wave 403 shown in FIG. 4. When low compliance is exhibited, less energy may be stored in the aorta compared to a healthy patient. Therefore, the systolic waveform 502 may demonstrate increased pressure during the systolic phase relative to a patient having normal compliance, while the diastolic waveform 501 may demonstrate reduced pressure during the diastolic phase relative to a patient having normal compliance. Therefore, the resulting combined waveform 503 may represent an increase in the systolic peak and a drop in the diastolic pressure, which may cause various health complications. For example, the change in waveform may impact the workload on the left ventricle and may adversely affect coronary profusion.

In view of the health complications that may be associated with reduced arterial compliance, as described above, it may be desirable in certain patients and/or under certain conditions, to at least partially alter compliance properties of the aorta or other artery or blood vessel, or otherwise alter/control flow therein, in order to improve cardiac/organ health. Disclosed herein are various devices and methods for at least partially restoring and/or increasing compliance in a blood vessel, such as the aorta. Certain examples disclosed herein achieve restoration of arterial compliance through the use of implantable and/or expandable implants configured to be implanted at least partially within a blood vessel and at least partially outside the blood vessel. For example, such implants may be configured to expand in accordance with elastic features/characteristics thereof and store energy during higher-pressure periods of the cardiac cycle (e.g., during the systolic phase). During lower-pressure periods (e.g., during the diastolic phase), such implant devices can contract/deflate to reshape the target blood vessel in a manner as to reduce a volume thereof to thereby return the stored energy to the circulation and increase flow through the vessel.

As the vasculature of a subject and/or of a subject blood vessel becomes less elastic, arterial blood pressure (e.g. left-ventricular afterload) can become more pulsatile. This can have deleterious effects such as thickening of the left ventricle (LV) muscle and/or diastolic heart failure.

In some examples, devices of the present disclosure include inflatable and/or expandable implants configured to be implant at least partially within a target blood vessel (e.g., the aorta) and/or at least partially outside the target blood vessel. A liquid-filled volume (e.g., implant and/or balloon) may be implanted partially within the aorta, and partly outside the aorta, such that, for each cycle (e.g., heartbeat), a distal and/or extraaortic portion of the liquid-filled volume can expand as increased aortic blood presses compresses a proximal and/or intraaortic portion. The distal and/or extraaortic portion of the volume can reinflate the proximal and/or intraaortic portion with the liquid as aortic blood pressure decreases. The volume and/or implant can therefore function as a hydraulic accumulator.

Devices of the present disclosure may be implanted at least partially within a target blood vessel, such as in the aorta (e.g., aortic trunk, descending thoracic, or abdominal aorta), using transcatheter and/or other minimally-invasive means, such as through a direct minimally-invasive path to the exterior of the aorta through the back and/or flank of the patient. With respect to transcatheter procedures, implants of the present disclosure may be advanced to the target area of the blood vessel through the vasculature.

Devices of the present disclosure may be delivered via the vasculature. In some examples, a branching blood vessel may be cut by deploying occluding devices prior to transecting the branching blood vessel completely (e.g., by an ablation) from within the vasculature rather than creating a minimally invasive access from, for example, the patient's back and/or flank.

### Compliance-Enhancing Hydraulic Accumulators

The present disclosure relates to systems, devices, and methods for adding back and/or increasing compliance in the aorta or other arterial (or venous) blood vessel(s) to provide improved perfusion of the heart muscle and/or other organ(s) of the body. Examples of the present disclosure can include inflatable and/or expandable implants which may be referred to herein as “accumulators” and/or “hydraulic accumulators.” The term “accumulator” is used herein in accordance with its plain and ordinary meaning and may refer to any balloon, container, bag, vessel, stent, and/or similar device configured to hold one or more gases and/or fluids. In some examples, an accumulator may be configured allow one or more contained gases and/or fluids to move between a proximal end and/or a distal end of the accumulator.

The accumulator can function as a hydraulic accumulator and/or may be configured to smooth out peaks (e.g., lower maximum blood pressure) and/or troughs (e.g., increase minimum blood pressure) in aortic blood pressure. A proximal and/or intraaortic part of the accumulator may be disposed between a wall of the aorta and a lined stent (e.g. similar to stents used for aortic aneurysms and/or parallel stent grafting). Increased blood pressure through the blood vessel can push against the lining of the stent, which can compress and/or deflate the proximal and/or intraaortic part. The stent may also advantageously provide sealing for the site at which the accumulator exits the blood vessel (e.g., aorta) via a hole in the aortic wall. The stent and/or accumulator may be configured to create hemostasis around the stent and/or accumulator and/or around the junction between the main blood vessel and the branching blood vessel.

The stent and/or accumulator may be configured to maintain hemostasis at either end of the stent and/or accumulator. In some examples, the stent may be configured to completely overlap, cover, and/or contain at least a portion of the accumulator. For example, the stent may fully enclose a proximal portion of the accumulator for hemostasis and/or positioning purposes inside the blood vessel.

In some examples, the accumulator may be sausage-shaped and/or may have wider/bulbous ends that displace a greater volume. The accumulator may comprise a balloon. At least a portion of the implant (e.g., a distal end and/or proximal end) may be at least partially elastic and/or may be configured to reinflate other portions of the accumulator in response to decreases in blood pressure.

Examples described herein relate to devices for smoothing out peaks and troughs in aortic blood pressure. Examples devices can be liquid-filled and/or can be configured for placement partially within the aorta and/or other blood vessel and/or partially outside the aorta and/or other blood vessel. In some examples, an implant may be configured to adjust and/or change on a periodic basis. For example, during each heartbeat, a distal portion of the implant (e.g., outside the aorta) may expand as increased blood pressure compresses a proximal portion (e.g., inside the aorta) and/or presses the liquid to the distal portion. Additionally or alternatively, the distal portion may be configured to reinflate the proximal portion with the liquid as blood pressure decreases.

The proximal and/or intraaortic portion of the device may be disposed between the wall of the aorta and a lined stent in some examples. The increased blood pressure within the aorta may pushes against the lining of the stent, which can compress the proximal and/or intraaortic portion. The stent may also advantageously provide sealing for the site at which the implant exits the aorta via a hole in the aortic wall.

As the implants of the present disclosure produce complaint blood vessel volume change by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a solution that avoids the risks that may be associated with cutting of the vessel and/or devices grafted in/to such vessels, which may present risk of rupture and blood leakage. Hazards associated with extravascular arterial blood leakage, such as within the abdominal and/or chest cavity, can include the risk of serious injury or death.

As described above, desirable diastolic flow in arterial blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel resisting stretching. Examples of the present disclosure provide implants that cause a change in cross-sectional area/volume of a target blood vessel without requiring stretching in the blood vessel wall. Rather, such cyclical change in blood vessel area/volume can be achieved through manipulation of the shape (e.g., cross-sectional shape) of the target blood vessel, wherein a transition between blood vessel shapes occurring in response to changing pressure conditions can reduce and increase the area/volume of the blood vessel in a cyclical manner to promote more even flow of blood through the blood vessel throughout the cardiac cycle.

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area of the blood vessel.

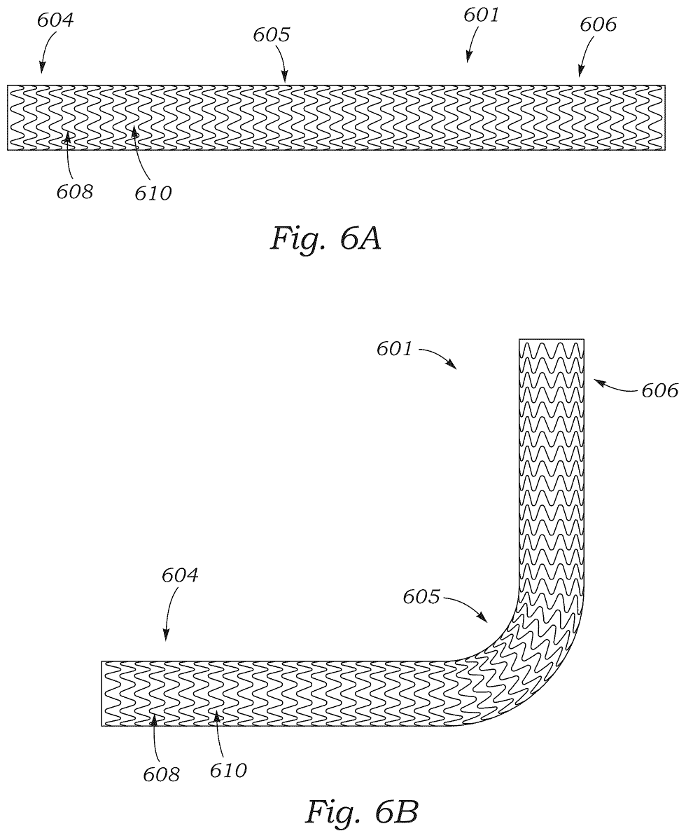

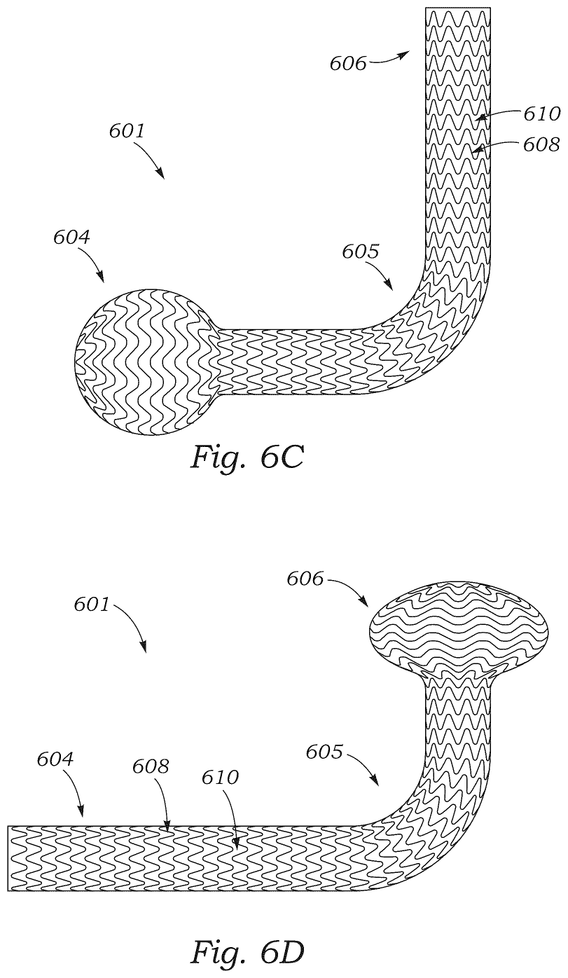

FIGS. 6A-6D illustrate an example implant 601 (e.g., hydraulic accumulator) configured to modulate blood pressures through one or more blood vessels of a heart in accordance with one or more examples. FIG. 6A illustrates the implant 601 in a generally tubular and/or cylindrical form, which the implant 601 may be configured to assume during and/or after delivery via a catheter and/or other delivery systems. FIG. 6B illustrates the implant 601 in an at least partially bent form, which the implant 601 may be configured to assume following delivery into a main blood vessel and/or into a branching blood vessel. FIG. 6C illustrates the implant 601 with a proximal portion 604 (e.g., intraaortic portion) at least partially inflated with gas and/or liquid. FIG. 6D illustrates the implant 601 with a distal portion 606 (e.g., extraaortic portion) at least partially inflated with gas and/or liquid.

The implant 601 may have a generally tubular and/or cylindrical shape and/or may be configured to assume a generally linear form during delivery to facilitate insertion of the implant 601 into a generally tubular catheter and/or similar device. In some examples, the implant 601 may comprise multiple components, which can include a wire frame 608 and/or a covering 610 (e.g., skirt). The frame 608 may comprise a network of struts configured to form one or more cells and/or openings through the frame 608. The covering 610 may be configured to at least partially enclose the one or more cells and/or to form a fluid-tight interior of the implant 601. For example, the covering 610 may comprise a pouch, balloon, and/or other container configured to retain gas and/or fluid without allowing leakage of the gas and/or fluid.

In some examples, the implant 601 may have a generally uniform diameter extending between a proximal portion 604 and a distal portion 606 of the implant 601. The proximal portion 604 and/or distal portion 606 may have generally uniform and/or constant diameters and/or may have increased diameters relative to a midsection 605 of the implant 601. The midsection 605 may comprise a portion of the implant 601 between the proximal portion 604 and the distal portion 606. In some examples, the proximal portion 604 and/or distal portion 606 may have generally wider and/or bulbous forms. For example, the proximal portion 604 may have a generally wider form as shown in FIG. 6C and/or the distal portion 606 may have a generally wider form as shown in FIG. 6D.

In some examples, the implant 601 and/or covering 610 may be configured to stretch and/or expand to allow for temporary increases in diameter of the proximal portion 604 and/or distal portion 606. At least a portion (e.g., the proximal portion 604 and/or distal portion 606) of the implant 601 may be at least partially elastic and/or may be configured to allow for transition of fluid in a seesaw and/or back-and-forth manner between the proximal portion 604 and the distal portion 606. In some examples, at least a portion of the implant 601 (e.g., the midsection 605) may be at least partially inelastic and/or may not be configured to expand and/or may be configured to maintain a default diameter. For example, the covering 610 may comprise different materials and/or a material at the midsection 605 may be generally rigid and/or inelastic. In some examples, the frame 608 may comprise multiple materials and/or may have generally reinforced portions (e.g., at the midsection 605) comprising relatively thick and/or densely situated struts. For example, struts at the midsection 605 may be densely spaced, may be generally thick, and/or may be at least partially composed of a generally rigid material and/or the frame 608 at the proximal portion 604 and/or distal portion 606 may comprise relatively spaced and/or thin struts and/or may be at least partially composed of a generally flexible and/or elastic material (e.g., Nitinol and/or other shape memory alloys.

The covering 610 may comprise one or more elastomers configured to provide an elasticity to the proximal portion 604 and/or distal portion 606. In some examples, the covering 610 and/or frame 608 may be generally inelastic and/or may be pressed back into a compressed form following expansion by a stent and/or other external devices.

The implant 601 may be configured for percutaneous delivery. In some examples, the implant 601 may be configured to smooth transitions between high blood pressure and low blood pressure within a blood vessel (e.g., the aorta). The implant 601 and/or the covering 610 may be configured to be filled with a gas and/or liquid. In some examples, the implant 601 may be filled following delivery and/or while disposed within a blood vessel and/or within a wall of the blood vessel. The implant 601 may be configured to extend at least partially out of the blood vessel. The distal portion 606 may be configured to be disposed outside the blood vessel and/or may be configured to elastically expand and/or fill with liquid. The proximal portion 604 may be configured to deflate in response to increased blood pressure inside the blood vessel and/or may be configured to inflate in response decreased blood pressure inside the blood vessel.

At least a portion (e.g., the distal portion 606) of the implant 601 may be configured to extend out of a blood vessel containing at least a portion (e.g., the proximal portion 604) of the implant 601. The implant 601 may advantageously be configured for filling with liquid, which are generally incompressible, and/or other incompressible fluids based at least in part on the implant 601 providing for the liquid to be pressed outside the blood vessel. Use of liquid may improve safety of the implant 601 and/or may cause a decreased likelihood of gradual loss of pressure from inflation fluid diffusing through walls of the implant 601 and/or covering 610.

The implant 601 may be configured to at least partially bend (e.g., to an approximately right angle and/or 90-degree bend) while disposed within a patient's body. For example, the implant 601 may be configured to bend at the midsection 605 and/or between the proximal portion 604 and the distal portion 606. Bending of the implant 601 may facilitate extension of the distal portion 606 out of a main blood vessel and/or into a branching blood vessel while the proximal portion 604 remains within the main blood vessel.

In some examples, the implant 601 may be configured to advantageously apply the Windkessel effect in restoring and/or improving compliance to one or more blood vessels. The implant 601 and/or the proximal portion 604 may be configured to press outwardly against a blood vessel during and/or after a pulse of the heart and/or other change in blood pressure. The implant 601 may then allow the blood vessel to return to a default size via transition of fluid through the implant 601.

Inflation and/or deflation of the proximal portion 604 and/or distal portion 606 may be performed passively, as a pulsatile action, and/or continuously in response to heart function. The implant 601 may operate similar to a spring. For example, the frame 608 may be at least partially composed of Nitinol and/or other shape memory alloy(s) and/or may be configured to bend and/or adjust in response to threshold forces. In some examples, the implant 601 may be responsive to particular and/or threshold blood pressure levels. For example, when blood pressure through the blood vessel increases above a threshold level (e.g., 100 mmHg), the implant 601 may be configured to quickly dilate and/or transfer fluid from the proximal portion 604 to the distal portion 606 to the form shown in FIG. 6C. Fluid may return from the distal portion 606 to the proximal portion 604 after the blood pressure drops below the threshold level.

In some examples, the proximal portion 604 and/or distal portion 606 may be configured to form bubbles and/or bulbs in response to increase of fluid. For example, fluid entering the proximal portion 604 distal portion 606 may cause the covering 610 to contract longitudinally and/or stretch/expand laterally to form a generally oval-shaped bulb. In some examples, the proximal portion 604 and/or distal portion 606 may be configured to form a generally ovular and/or non-circular bulb. The covering 610 and/or frame 608 may be shape set to control expansion of the proximal portion 604 and/or distal portion 606 to any desired size and/or shape.

The frame 608 of the implant 601 can be shape set to any desired shape. In some examples, the frame 608 may be shape set to form an approximately 90-degree bend, as shown in FIGS. 6B-6D. Moreover, the frame 608 may be shape set to naturally form a bulb at the proximal portion 604. The shape set form of the frame 608 may be configured to be overcome by increased blood pressure through the blood vessel. In some examples, the covering 610 can be shape set to the form shown in FIG. 6B and/or FIG. 6C. The covering 610 can comprise any suitable material and/or may be at least partially composed of polymer, rubber, and/or other materials. The covering 610 may be configured to elastically stretch and/or expand.

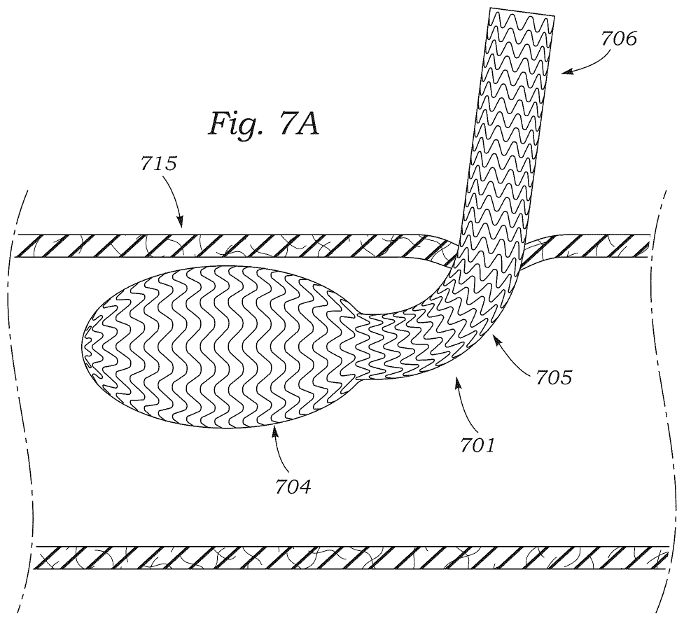

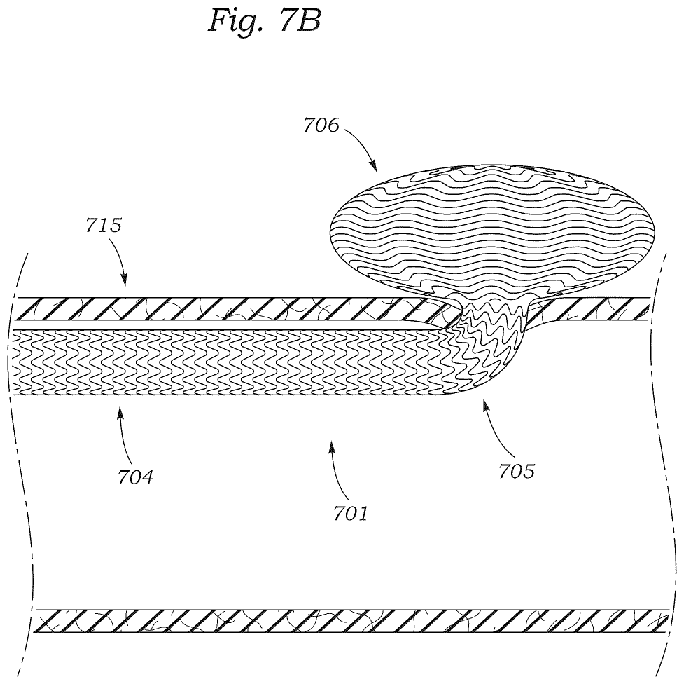

FIGS. 7A and 7B illustrate an example implant 701 (e.g., accumulator) implanted in a blood vessel 715 (e.g., an aorta) to modulate blood pressures through the blood vessel 715 in accordance with one or more examples. FIG. 7A illustrates the implant 701 with a proximal portion 704 (e.g., intraaortic portion) at least partially inflated with gas and/or liquid disposed within a covering and/or frame of the implant 701. FIG. 7D illustrates the implant 701 with a distal portion 706 (e.g., extraaortic portion) at least partially inflated with gas and/or liquid. The proximal portion 704 may be configured to be disposed within the blood vessel 715 and/or in contact with an inner wall of the blood vessel 715 and/or the distal portion 706 may be configured to be disposed outside the blood vessel 715 and/or in contact with an outer wall of the blood vessel 715. A midsection 705 of the implant 701 may be configured to extend through an opening in the blood vessel 715.

The implant 701 may have a generally tubular and/or cylindrical shape and/or may be configured to assume a generally linear form during delivery to facilitate insertion of the implant 701 into a generally tubular catheter and/or similar device. In some examples, the implant 701 may comprise multiple components, which can include a wire frame and/or a covering (e.g., skirt). The frame may comprise a network of struts configured to form one or more cells and/or openings through the frame. The covering may be configured to at least partially enclose the one or more cells and/or to form a fluid-tight interior of the implant 701. For example, the covering may comprise a pouch, balloon, and/or other container configured to retain gas and/or fluid without allowing leakage of the gas and/or fluid.

In some examples, the implant 701 may have a generally uniform diameter extending between a proximal portion 704 and a distal portion 706 of the implant 701. The proximal portion 704 and/or distal portion 706 may have generally uniform and/or constant diameters and/or may have increased diameters relative to a midsection 705 of the implant 701. The midsection 705 may comprise a portion of the implant 701 between the proximal portion 704 and the distal portion 706. In some examples, the proximal portion 704 and/or distal portion 706 may have generally wider and/or bulbous forms. For example, the proximal portion 704 may have a generally wider form as shown in FIG. 7A and/or the distal portion 706 may have a generally wider form as shown in FIG. 7B.

In some examples, the implant 701 and/or covering may be configured to stretch and/or expand to allow for temporary increases in diameter of the proximal portion 704 and/or distal portion 706. At least a portion (e.g., the proximal portion 704 and/or distal portion 706) of the implant 701 may be at least partially elastic and/or may be configured to allow for transition of fluid in a seesaw and/or back-and-forth manner between the proximal portion 704 and the distal portion 706. In some examples, at least a portion of the implant 701 (e.g., the midsection 705) may be at least partially inelastic and/or may not be configured to expand and/or may be configured to maintain a default diameter. For example, the covering may comprise different materials and/or a material at the midsection 705 may be generally rigid and/or inelastic. In some examples, the frame may comprise multiple materials and/or may have generally reinforced portions (e.g., at the midsection 705) comprising relatively thick and/or densely situated struts. For example, struts at the midsection 705 may be densely spaced, may be generally thick, and/or may be at least partially composed of a generally rigid material and/or the frame at the proximal portion 704 and/or distal portion 706 may comprise relatively spaced and/or thin struts and/or may be at least partially composed of a generally flexible and/or elastic material (e.g., Nitinol and/or other shape memory alloys.

The covering may comprise one or more elastomers configured to provide an elasticity to the proximal portion 704 and/or distal portion 706. In some examples, the covering and/or frame may be generally inelastic and/or may be pressed back into a compressed form following expansion by a stent and/or other external devices.

The implant 701 may be configured for percutaneous delivery. In some examples, the implant 701 may be configured to smooth transitions between high blood pressure and low blood pressure within a blood vessel (e.g., the aorta). The implant 701 and/or the covering may be configured to be filled with a gas and/or liquid. In some examples, the implant 701 may be filled following delivery and/or while disposed within a blood vessel and/or within a wall of the blood vessel. The implant 701 may be configured to extend at least partially out of the blood vessel. The distal portion 706 may be configured to be disposed outside the blood vessel and/or may be configured to elastically expand and/or fill with liquid. The proximal portion 704 may be configured to deflate in response to increased blood pressure inside the blood vessel and/or may be configured to inflate in response decreased blood pressure inside the blood vessel.

At least a portion (e.g., the distal portion 706) of the implant 701 may be configured to extend out of a blood vessel containing at least a portion (e.g., the proximal portion 704) of the implant 701. The implant 701 may advantageously be configured for filling with liquid, which are generally incompressible, and/or other incompressible fluids based at least in part on the implant 701 providing for the liquid to be pressed outside the blood vessel. Use of liquid may improve safety of the implant 701 and/or may cause a decreased likelihood of gradual loss of pressure from inflation fluid diffusing through walls of the implant 701 and/or covering.

The implant 701 may be configured to at least partially bend (e.g., to an approximately right angle and/or 90-degree bend) while disposed within a patient's body. For example, the implant 701 may be configured to bend at the midsection 705 and/or between the proximal portion 704 and the distal portion 706. Bending of the implant 701 may facilitate extension of the distal portion 706 out of a main blood vessel and/or into a branching blood vessel while the proximal portion 704 remains within the main blood vessel.

In some examples, the implant 701 may be configured to advantageously apply the Windkessel effect in restoring and/or improving compliance to one or more blood vessels. The implant 701 and/or the proximal portion 704 may be configured to press outwardly against a blood vessel during and/or after a pulse of the heart and/or other change in blood pressure. The implant 701 may then allow the blood vessel to return to a default size via transition of fluid through the implant 701.

Inflation and/or deflation of the proximal portion 704 and/or distal portion 706 may be performed passively, as a pulsatile action, and/or continuously in response to heart function. The implant 701 may operate similar to a spring. For example, the frame may be at least partially composed of Nitinol and/or other shape memory alloy(s) and/or may be configured to bend and/or adjust in response to threshold forces. In some examples, the implant 701 may be responsive to particular and/or threshold blood pressure levels. For example, when blood pressure through the blood vessel increases above a threshold level (e.g., 100 mmHg), the implant 701 may be configured to quickly dilate and/or transfer fluid from the proximal portion 704 to the distal portion 706 to the form shown in FIG. 7C. Fluid may return from the distal portion 706 to the proximal portion 704 after the blood pressure drops below the threshold level.

In some examples, the proximal portion 704 and/or distal portion 706 may be configured to form bubbles and/or bulbs in response to increase of fluid. For example, fluid entering the proximal portion 704 distal portion 706 may cause the covering to contract longitudinally and/or stretch/expand laterally to form a generally oval-shaped bulb. In some examples, the proximal portion 704 and/or distal portion 706 may be configured to form a generally ovular and/or non-circular bulb. The covering and/or frame may be shape set to control expansion of the proximal portion 704 and/or distal portion 706 to any desired size and/or shape.

The frame of the implant 701 can be shape set to any desired shape. In some examples, the frame may be shape set to form an approximately 90-degree bend, as shown in FIGS. 7A and 7B. Moreover, the frame may be shape set to naturally form a bulb at the proximal portion 704. The shape set form of the frame may be configured to be overcome by increased blood pressure through the blood vessel. In some examples, the covering can be shape set to the form shown in FIG. 7B and/or FIG. 7C. The covering can comprise any suitable material and/or may be at least partially composed of polymer, rubber, and/or other materials. The covering may be configured to elastically stretch and/or expand.

In some examples, the distal portion 706 may be configured to be crimped and/or collapsed initially to drive fluid volume into the vessel 715 and/or towards the proximal portion 704. The implant 701 may have a fluid transfer orifice (e.g., lumen) of approximately 4-6 mm in diameter. In some examples, the implant 701 may be configured to transfer approximately 10 ml between diastole and systole pressures. The implant 701 may be fluid-tight and/or may be configured to be completely sealed and/or to prevent leakage.

The implant 701 may provide a pulsatile operation in which, in each working cycle, the inner portion of the implant 701 may be configured to deflate at a defined pressure and/or inflate back in low pressure (e.g., less than 90 mmHg).

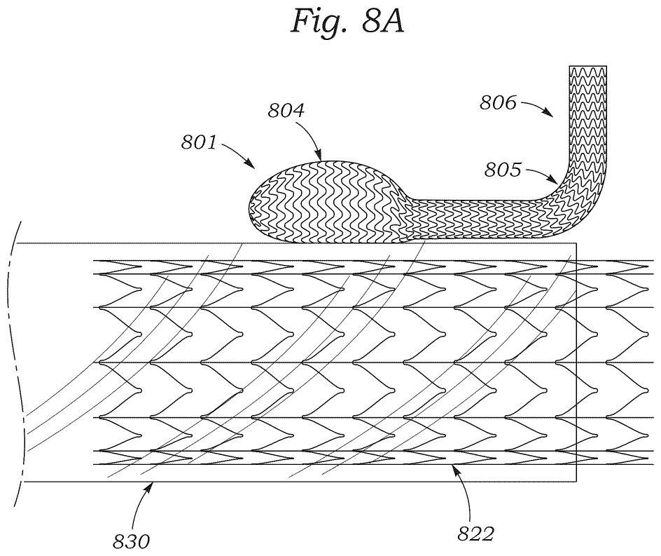

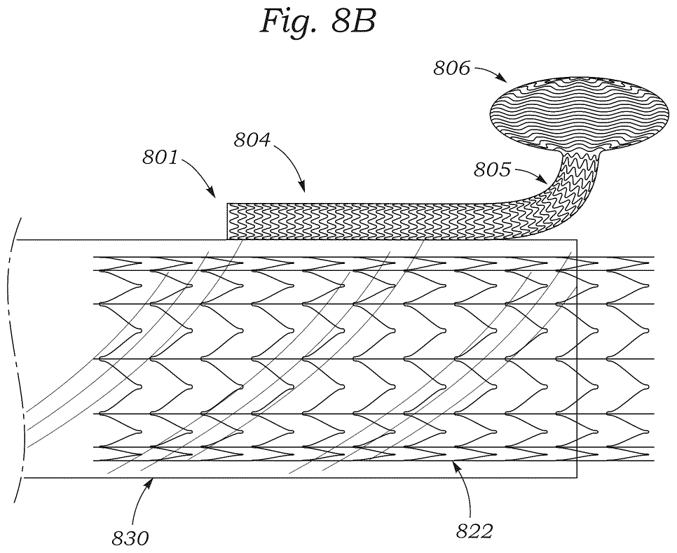

FIGS. 8A and 8B illustrate an example implant 801 (e.g., accumulator) and stent 822 configured to modulate blood pressures through one or more blood vessels of a heart in accordance with one or more examples. FIG. 8A illustrates the implant 801 with a proximal portion 804 (e.g., intraaortic portion) at least partially inflated with gas and/or liquid disposed within a covering and/or frame of the implant 801. FIG. 8D illustrates the implant 801 with a distal portion 806 (e.g., extraaortic portion) at least partially inflated with gas and/or liquid. The proximal portion 804 may be configured to be disposed within the blood vessel and/or in contact with an inner wall of the blood vessel and/or the distal portion 806 may be configured to be disposed outside the blood vessel and/or in contact with an outer wall of the blood vessel. A midsection 805 of the implant 801 may be configured to extend through an opening in the blood vessel.

The implant 801 may have a generally tubular and/or cylindrical shape and/or may be configured to assume a generally linear form during delivery to facilitate insertion of the implant 801 into a generally tubular catheter and/or similar device. In some examples, the implant 801 may comprise multiple components, which can include a wire frame and/or a covering (e.g., skirt). The frame may comprise a network of struts configured to form one or more cells and/or openings through the frame. The covering may be configured to at least partially enclose the one or more cells and/or to form a fluid-tight interior of the implant 801. For example, the covering may comprise a pouch, balloon, and/or other container configured to retain gas and/or fluid without allowing leakage of the gas and/or fluid.

In some examples, the implant 801 may have a generally uniform diameter extending between a proximal portion 804 and a distal portion 806 of the implant 801. The proximal portion 804 and/or distal portion 806 may have generally uniform and/or constant diameters and/or may have increased diameters relative to a midsection 805 of the implant 801. The midsection 805 may comprise a portion of the implant 801 between the proximal portion 804 and the distal portion 806. In some examples, the proximal portion 804 and/or distal portion 806 may have generally wider and/or bulbous forms. For example, the proximal portion 804 may have a generally wider form as shown in FIG. 8A and/or the distal portion 806 may have a generally wider form as shown in FIG. 8B.

In some examples, the implant 801 and/or covering may be configured to stretch and/or expand to allow for temporary increases in diameter of the proximal portion 804 and/or distal portion 806. At least a portion (e.g., the proximal portion 804 and/or distal portion 806) of the implant 801 may be at least partially elastic and/or may be configured to allow for transition of fluid in a seesaw and/or back-and-forth manner between the proximal portion 804 and the distal portion 806. In some examples, at least a portion of the implant 801 (e.g., the midsection 805) may be at least partially inelastic and/or may not be configured to expand and/or may be configured to maintain a default diameter. For example, the covering may comprise different materials and/or a material at the midsection 805 may be generally rigid and/or inelastic. In some examples, the frame may comprise multiple materials and/or may have generally reinforced portions (e.g., at the midsection 805) comprising relatively thick and/or densely situated struts. For example, struts at the midsection 805 may be densely spaced, may be generally thick, and/or may be at least partially composed of a generally rigid material and/or the frame at the proximal portion 804 and/or distal portion 806 may comprise relatively spaced and/or thin struts and/or may be at least partially composed of a generally flexible and/or elastic material (e.g., Nitinol and/or other shape memory alloys.

The covering may comprise one or more elastomers configured to provide an elasticity to the proximal portion 804 and/or distal portion 806. In some examples, the covering and/or frame may be generally inelastic and/or may be pressed back into a compressed form following expansion by a stent 822 and/or other external devices.

The implant 801 may be configured for percutaneous delivery. In some examples, the implant 801 may be configured to smooth transitions between high blood pressure and low blood pressure within a blood vessel (e.g., the aorta). The implant 801 and/or the covering may be configured to be filled with a gas and/or liquid. In some examples, the implant 801 may be filled following delivery and/or while disposed within a blood vessel and/or within a wall of the blood vessel. The implant 801 may be configured to extend at least partially out of the blood vessel. The distal portion 806 may be configured to be disposed outside the blood vessel and/or may be configured to elastically expand and/or fill with liquid. The proximal portion 804 may be configured to deflate in response to increased blood pressure inside the blood vessel and/or may be configured to inflate in response decreased blood pressure inside the blood vessel.

At least a portion (e.g., the distal portion 806) of the implant 801 may be configured to extend out of a blood vessel containing at least a portion (e.g., the proximal portion 804) of the implant 801. The implant 801 may advantageously be configured for filling with liquid, which are generally incompressible, and/or other incompressible fluids based at least in part on the implant 801 providing for the liquid to be pressed outside the blood vessel. Use of liquid may improve safety of the implant 801 and/or may cause a decreased likelihood of gradual loss of pressure from inflation fluid diffusing through walls of the implant 801 and/or covering.

The implant 801 may be configured to at least partially bend (e.g., to an approximately right angle and/or 90-degree bend) while disposed within a patient's body. For example, the implant 801 may be configured to bend at the midsection 805 and/or between the proximal portion 804 and the distal portion 806. Bending of the implant 801 may facilitate extension of the distal portion 806 out of a main blood vessel and/or into a branching blood vessel while the proximal portion 804 remains within the main blood vessel.

In some examples, the implant 801 may be configured to advantageously apply the Windkessel effect in restoring and/or improving compliance to one or more blood vessels. The implant 801 and/or the proximal portion 804 may be configured to press outwardly against a blood vessel during and/or after a pulse of the heart and/or other change in blood pressure. The implant 801 may then allow the blood vessel to return to a default size via transition of fluid through the implant 801.

Inflation and/or deflation of the proximal portion 804 and/or distal portion 806 may be performed passively, as a pulsatile action, and/or continuously in response to heart function. The implant 801 may operate similar to a spring. For example, the frame may be at least partially composed of Nitinol and/or other shape memory alloy(s) and/or may be configured to bend and/or adjust in response to threshold forces. In some examples, the implant 801 may be responsive to particular and/or threshold blood pressure levels. For example, when blood pressure through the blood vessel increases above a threshold level (e.g., 100 mmHg), the implant 801 may be configured to quickly dilate and/or transfer fluid from the proximal portion 804 to the distal portion 806 to the form shown in FIG. 8C. Fluid may return from the distal portion 806 to the proximal portion 804 after the blood pressure drops below the threshold level.

In some examples, the proximal portion 804 and/or distal portion 806 may be configured to form bubbles and/or bulbs in response to increase of fluid. For example, fluid entering the proximal portion 804 distal portion 806 may cause the covering to contract longitudinally and/or stretch/expand laterally to form a generally oval-shaped bulb. In some examples, the proximal portion 804 and/or distal portion 806 may be configured to form a generally ovular and/or non-circular bulb. The covering and/or frame may be shape set to control expansion of the proximal portion 804 and/or distal portion 806 to any desired size and/or shape.

The frame of the implant 801 can be shape set to any desired shape. In some examples, the frame may be shape set to form an approximately 90-degree bend, as shown in FIGS. 8A and 8B. Moreover, the frame may be shape set to naturally form a bulb at the proximal portion 804. The shape set form of the frame may be configured to be overcome by increased blood pressure through the blood vessel. In some examples, the covering can be shape set to the form shown in FIG. 8B and/or FIG. 8C. The covering can comprise any suitable material and/or may be at least partially composed of polymer, rubber, and/or other materials. The covering may be configured to elastically stretch and/or expand.

At least a portion of the implant 801 may be configured for placement adjacent to a stent 822. The stent 822 may comprise a wire frame and/or a network of struts forming one or more cells and/or openings through the stent 822. In some examples, the stent 822 may be at least partially enclosed by a covering 830. For example, the covering 830 may be adhered to the frame 822 through heat treatment and/or other suitable means.

The stent 822 may have a generally cylindrical and/or tubular form. In some examples, the stent 822 may be sized and/or shaped to approximate a size and/or shape of a blood vessel of a heart.

At least a portion of the implant 801 (e.g., the proximal portion 804) may be configured to be disposed at least partially between a wall of a blood vessel and the stent 822. The proximal portion 804 may be configured to be inflated into a generally ovular bulb between the stent 822 and the vessel wall in a default state. As blood pressure through the blood vessel increases, the walls/sides of the stent 822 may be pressed outwardly and/or may be configured to compress the proximal portion 804 and/or to press fluid from the proximal portion 804 to the distal portion 806 to inflate the distal portion 806, as shown in FIG. 8B.

In some examples, the stent 822 and/or covering 830 may be configured to at least partially cover and/or seal an opening in the vessel wall and/or an inflow junction of one or more branching blood vessels. For example, the stent 822 and/or covering 830 may be configured to at least partially block and/or occlude an opening through which the implant 801 (e.g., a midsection of the implant 801) extends.

The stent 822 may be configured to self-expand and/or expand through balloon and/or other means to contact the vessel walls. For example, there may be no blood flow between walls of the stent 822 and the vessel walls. The stent 822 may have a variable and/or uneven diameter across a length of the stent 822 and/or may be shaped to approximate a shape of a blood vessel. In some examples, the stent 822 may have a greater diameter than the implant 801.

In some examples, the implant 801 may be coupled to the stent 822 and/or covering 830 to avoid migration of the implant 801 and/or stent 822 relative to each other. For example, the implant 801 may be glued, stitched, and/or otherwise adhered to the stent 822. In some examples, the stent 822 may comprise one or more anchors configured to attach to the implant 801.

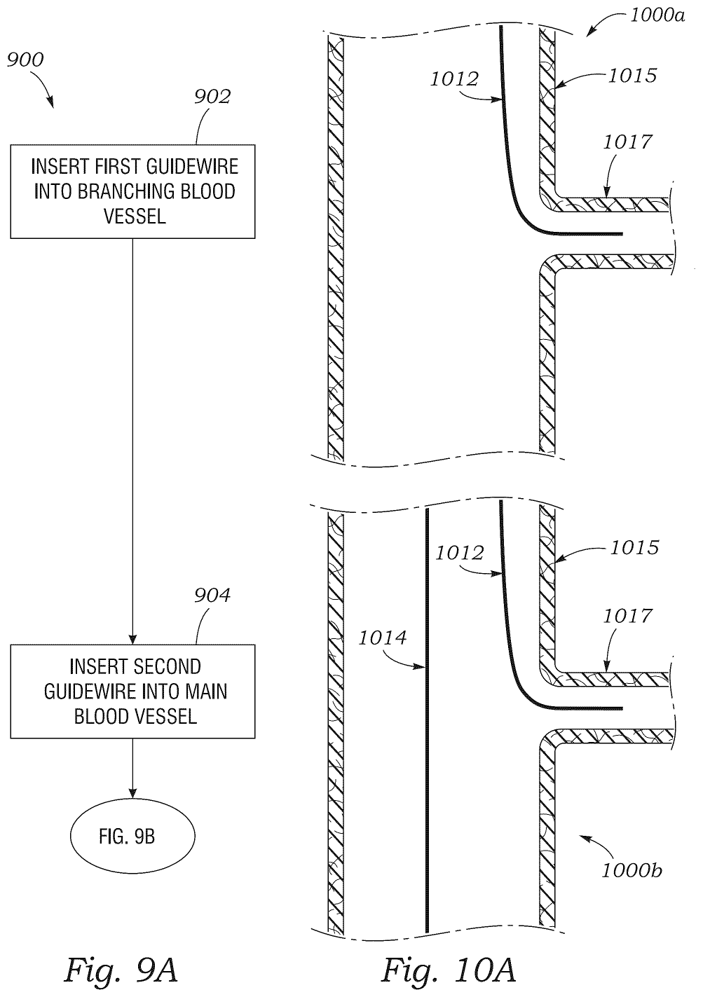

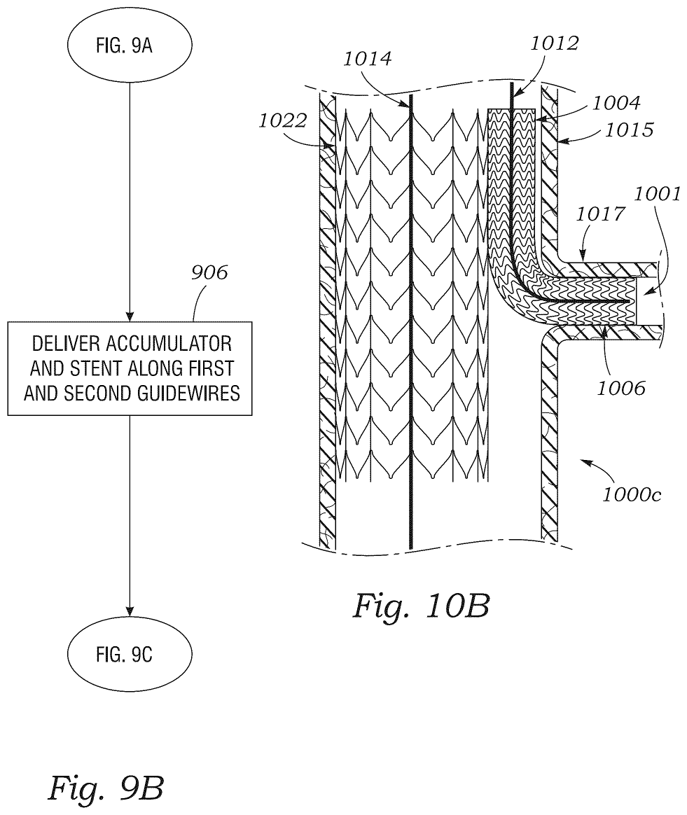

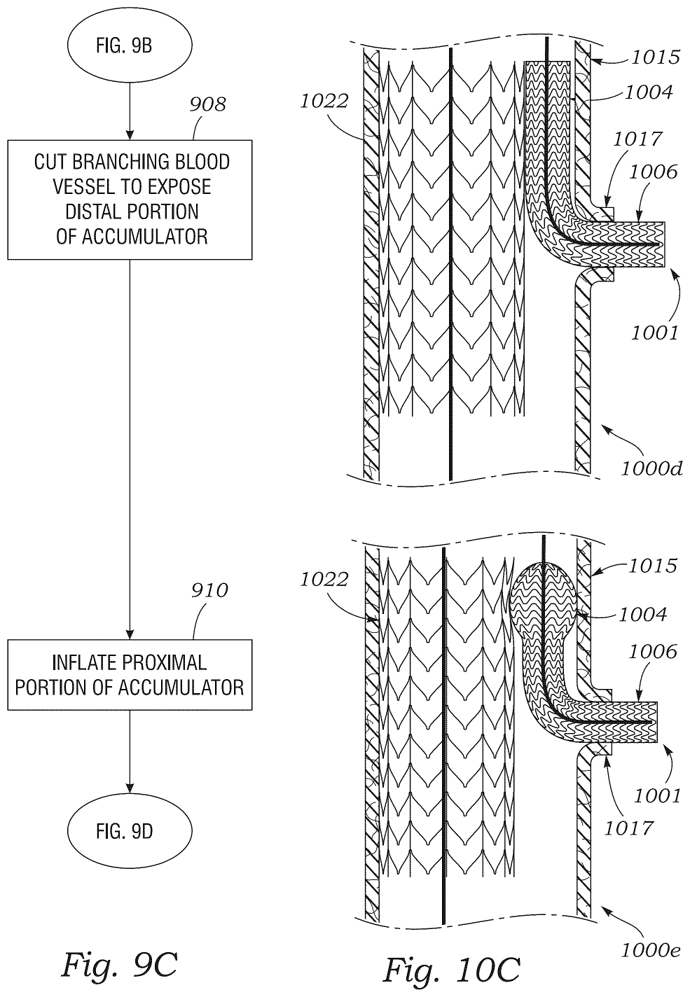

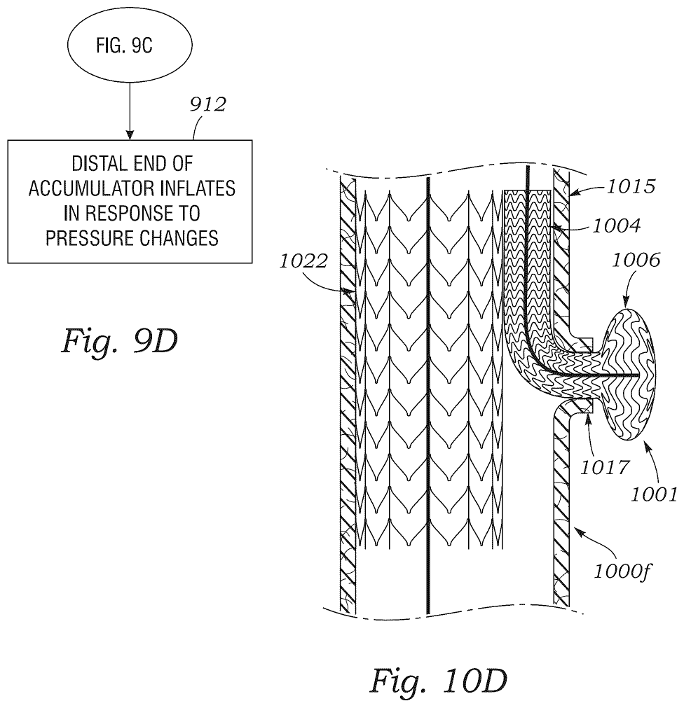

FIGS. 9A-9D illustrate a flow diagram for a process 900 delivering and/or implanting a compliance-enhancing system including one or more accumulators 1001 (e.g., inflatable implants) and/or stents 1022 in accordance with one or more examples. FIGS. 10A-10D provide images of the compliance-enhancing system and certain anatomy corresponding to operations of the process 900 of FIGS. 9A-9D according to one or more examples.

At step 902, the process 900 involves inserting a first guidewire 1012 via a main blood vessel 1015 (e.g., the aorta) into a branching blood vessel 1017 (e.g., an intercostal blood vessel), as illustrated in image 1000a of FIG. 10A. In some examples, the branching blood vessel 1017 may extend generally perpendicularly from the main blood vessel 1015. Alternatively, the process 900 may involve creating an opening in a wall of the main blood vessel 1015 and/or passing the first guidewire 1012 through the opening.

The main blood vessel 1015 (e.g., the aorta) and/or branching blood vessel 1017 may be accessed percutaneously and/or in any suitable manner. For example, access to the aorta may be achieved via a transfemoral delivery. The first guidewire 1012 may be navigated first into the main blood vessel 1015 and then into the branching blood vessel 1017. The branching blood vessel 1017 may have a smaller diameter than the main blood vessel 1015. For example, the branching blood vessel 1017 may have a width of approximately 1 mm. In some examples, the branching vessel 1017 can be expanded from approximately 1 mm to approximately 6 mm using, for example, an expansion balloon with a diameter of approximately 8 mm. In some examples, the process 900 may involve expanding the inner diameter of the branching blood vessel 1017 and/or main blood vessel 1015.

At step 904, the process 900 involves inserting a second guidewire 1014 into the main blood vessel 1015, as shown in image 1000b of FIG. 10A. The first guidewire 1012 may remain in place while the second guidewire 1014 is delivered. The second guidewire 1014 may not be inserted into the branching blood vessel 1017. The second guidewire 1014 may be delivered to the main blood vessel 1015 via the femoral vein.

At step 906, the process 900 involves delivering an accumulator 1001 over the first guidewire 1012 and/or delivering a stent 1022 over the second guidewire 1014, as shown in image 1000c of FIG. 10B. In some examples, the accumulator 1001 and the stent 1022 may be delivered simultaneously and/or in series. For example, the stent 1022 may be delivered into the main blood vessel 1015 first, such that the first guidewire 1012 may be sandwiched between the stent 1022 and the walls of the main blood vessel 1015. The stent 1022 may comprise a wire frame and/or may be at least partially enclosed by a covering.

The accumulator 1001 may be extended at least partially into the branching blood vessel 1017. In some examples, the accumulator 1001 may be configured to be disposed partially within the branching blood vessel 1017 and partially within the main blood vessel 1015, as shown in image 1000c.

In some examples, the accumulator 1001 may be delivered in a deflated and/or compressed form. For example, the accumulator 1001 may be delivered without any fluid and/or with minimal fluid inside the accumulator 1001. The accumulator 1001 may be attached to one or more tubes configured to convey fluids into the accumulator 1001 following delivery and/or placement of the accumulator 1001. Alternatively, the accumulator 1001 may be delivered in an inflated and/or at least partially expanded form.

The stent 1022 may be delivered in a compressed form. For example, the stent 1022 may be configured to assume a smaller width and/or diameter during delivery. The stent 1022 may be configured to self-expand to the shape set form shown in image 1000c following removal from a catheter and/or other delivery device and/or may be expanded via balloon expansion and/or other suitable means.

While the stent 1022 is shown forming one or more gaps between the stent 1022 and the walls of the vessel 1015, the stent 1022 and/or accumulator 1001 may be configured to fill empty space and/or to flexibly bend around each other to create hemostasis around the stent 1022 and/or accumulator 1001. The stent 1022 and/or accumulator 1001 may be configured to form two tiers of hemostasis proximal and/or distal to the stent 1022 and/or accumulator 1001 and/or between the stent 1022 and/or accumulator 1001 and the walls of the vessel 1015. In some examples, the stent 1022 and/or accumulator 1001 may be at least partially over-sized to balloon outwardly and/or to fill empty space. The stent 1022 may be configured to extend beyond the accumulator 1001 and/or may bend around an outer surface of the accumulator 1001.

At step 908, the process 900 involves cutting and/or severing the branching blood vessel 1017 to at least partially expose at least portion (e.g., the distal portion 1006) of the accumulator 1001, as shown in image 1000d of FIG. 10C. In embodiments in which the first guidewire 1012 is passed through an opening in the walls of the main blood vessel 1015, cutting may not be necessary as the first guidewire 1012 and/or the distal portion 1006 of the accumulator 1001 may be exposed outside the main blood vessel 1015. The distal portion 1006 may be disposed generally in a thoracic space and/or within a chamber of a heart and/or may have room for expansion beyond the main blood vessel 1015.

The branching blood vessel 1017 may be cut by any suitable means. In some examples, the branching blood vessel 1017 may be at least partially occluded prior to cutting to reduce risk of an aneurysm. The branching blood vessel 1017 may be cut from the inside or from the outside using, for example, an ablation 360 tool.

At step 910, the process 900 involves inflating a proximal portion 1004 (e.g., proximal end) of the accumulator 1001, as shown in image 1000e of FIG. 10C. In some examples, a fluid (e.g., saline) may be slowly injected and/or delivered into the accumulator 1001 via tubes and/or similar devices. In some examples, fluid tubes may be disconnected from the accumulator 1001 following inflation of the proximal portion 1004. The accumulator 1001 may be monitored during inflation to calibrate a desired level of inflation. In some examples, the accumulator 1001 and/or a frame and/or covering of the implant may be shape set such that inflated fluid initially enters the proximal portion 1004 and/or such that the distal portion 1006 remains generally deflated.

The accumulator 1001 may be shape set such that the proximal portion 1004 may be configured to inflate and/or expand in width in response to injection and/or delivery of fluid into the accumulator 1001. For example, the proximal portion 1004 may be shape set into a bulb form and/or may be shape set to a larger width and/or diameter than the distal portion 1006. In another example, the proximal portion 1004 may comprise relatively highly elastic materials and/or may be configured to more easily expand than the distal portion 1006.

The proximal portion 1004 and/or distal portion 1006 may be configured to inflate and/or expand in response to receiving injected fluid. In some examples, the proximal portion 1004 may be configured to inflate and/or expand in response to injection of fluid into the accumulator 1001 and/or in response to receiving fluid. The distal portion 1004 may be configured to remain uninflated and/or unexpanded following injection of fluid into the accumulator 1001. For example, the distal portion 1006 may receive fluid but the fluid may not inflate and/or expand distal portion 1006 until a sufficient amount of fluid and/or a sufficiently pressurized fluid is received at the distal portion 1006 due to, for example, relatively rigid materials at the distal portion 1006 and/or the fluid filling a bulb-like expansion of the proximal portion 1004. An increase of fluid and/or fluid pressure received at the distal portion 1006 (e.g., caused by deflation and/or compression of the proximal portion 1004 in response to changes in blood pressure of the vessel 1015) may cause the distal portion 1006 to inflate and/or expand to a bulb and/or disc shape. The proximal portion 1004 and/or distal portion 1006 may be shape set to form various shapes in response to expansion caused by fluid. For example, the proximal portion 1004 may be configured to form a bulb and/or disc shape and/or the distal portion 1006 may be configured to form a bulb and/or disc shape.

As the proximal portion 1004 inflates, the proximal portion 1004 may press against the stent 1022 and/or walls of the main blood vessel 1015. In some examples, inflation of the proximal portion 1004 may cause an increase in the width and/or diameter of the main blood vessel 1015.

At step 912, the process 900 involves the distal portion 1006 (e.g., distal end) passively and/or actively inflating in response to pressure changes within the main blood vessel 1015, as shown in image 1000f of FIG. 10D. The proximal portion 1004 may be configured to deflate and/or press fluid to the distal portion 1006 in response to increased pressure within the main blood vessel 1015. For example, the proximal portion 1004 may be configured to deflate in response to blood pressure above approximately 90 mmHg (e.g., higher than diastolic pressure) and/or below approximately 160 mmHg (e.g., lower than systolic pressure).

The above method(s) can be performed on a living animal or on a simulation, such as on a cadaver, cadaver heart, anthropomorphic ghost, simulator (e.g., with body parts, heart, tissue, etc. being simulated).

### Additional Description of Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A compliance-restoration system comprising an implant configured to be inflated with a fluid, the implant having a proximal portion and a distal portion, the proximal portion and the distal portion configured to expand in width in response to receiving fluid, wherein the distal portion is configured to inflate with the fluid in response to deflation of the proximal portion.

Example 2: The compliance-restoration system of any example herein, in particular example 1, wherein the implant is shape set to form an approximately 90-degree angle.

Example 3: The compliance-restoration system of any example herein, in particular example 1, wherein the implant comprises a wire frame and a covering.

Example 4: The compliance-restoration system of any example herein, in particular example 3, wherein the wire frame is shape set to form a bulb at the proximal portion in response to an influx of fluid at the proximal portion.

Example 5: The compliance-restoration system of any example herein, in particular example 3, wherein the wire frame is shape set to form a bulb at the distal portion in response to an influx of fluid at the proximal portion.

Example 6: The compliance-restoration system of any example herein, in particular example 1, wherein the implant is configured for placement at least partially within a blood vessel.

Example 7: The compliance-restoration system of any example herein, in particular example 6, wherein the distal portion is configured to extend out of the blood vessel.

Example 8: The compliance-restoration system of any example herein, in particular example 6, wherein the proximal portion is configured to compress to press fluid to the distal portion in response to an increase of blood pressure in the blood vessel.

Example 9: The compliance-restoration system of any example herein, in particular example 6, wherein proximal portion is configured to compress in response to blood pressure in the blood vessel being above diastolic pressure.

Example 10: The compliance-restoration system of any example herein, in particular example 1, wherein the proximal portion is configured to form an oval-shaped bulb in response to receiving fluid.

Example 11: The compliance-restoration system of any example herein, in particular example 1, wherein the distal portion is configured to form a disc-shaped bulb in response to receiving fluid.

Example 12: The compliance-restoration system of any example herein, in particular example 1, wherein the proximal portion is configured to inflate and the distal portion is configured to not inflate in response to injection of fluid into the implant.

Example 13: The compliance-restoration system of any example herein, in particular example 1, further comprising a stent, wherein the implant is configured for placement at least partially between the stent and an inner wall of a blood vessel.

Example 14: The compliance-restoration system of any example herein, in particular example 13, wherein the stent comprises a wire frame and a covering.

Example 15: The compliance-restoration system of any example herein, in particular example 14, wherein the implant has a smaller diameter than the stent.

Example 16: The compliance-restoration system of any example herein, in particular example 14, wherein the stent is configured to press against the proximal portion to cause deflation of the proximal portion in response to increased blood pressure at the stent.

Example 17: A method comprising percutaneously delivering an implant into a first blood vessel; extending a distal portion of the implant out of the first blood vessel while retaining a proximal portion of the implant within the first blood vessel; and injecting the implant with a fluid, wherein the proximal portion is configured to inflate in response to injection of the fluid, and wherein the distal portion is configured to inflate in response to deflation of the proximal portion.

Example 18: The method of any example herein, in particular example 17, wherein the implant is shape set to form an approximately 90-degree angle.

Example 19: The method of any example herein, in particular example 17, wherein the implant comprises a wire frame and a covering.

Example 20: The method of any example herein, in particular example 19, wherein the wire frame is shape set to form a bulb at the proximal portion in response to inflation of the fluid.

Example 21: The method of any example herein, in particular example 19, wherein the wire frame is shape set to form a bulb at the distal portion in response to the fluid being pushed out of the proximal portion.

Example 22: The method of any example herein, in particular example 17, wherein the proximal portion is configured to form an oval-shaped bulb in response to receiving fluid.

Example 23: The method of any example herein, in particular example 17, wherein the distal portion is configured to form a disc-shaped bulb in response to receiving fluid.

Example 24: The method of any example herein, in particular example 17, wherein the proximal portion is configured to inflate and the distal portion is configured to not inflate in response to injection of fluid into the implant.

Example 25: The method of any example herein, in particular example 17, wherein the proximal portion is configured to deflate in response to an increase of blood pressure in the first blood vessel.

Example 26: The method of any example herein, in particular example 17, wherein the proximal portion is configured to deflate in response to blood pressure in the first blood vessel being above diastolic pressure, and wherein the proximal portion is configured to inflate in response to deflation of the distal portion.

Example 27: The method of any example herein, in particular example 17, further comprising a stent, wherein the implant is configured for placement at least partially between the stent and an inner wall of the first blood vessel.

Example 28: The method of any example herein, in particular example 27, wherein the stent comprises a wire frame and a covering.

Example 29: The method of any example herein, in particular example 27, wherein the implant has a smaller diameter than the stent.

Example 30: The method of any example herein, in particular example 27, wherein the stent is configured to press against the proximal portion to cause deflation of the proximal portion in response to increased blood pressure at the stent.

Example 31: The method of any example herein, in particular example 17, further comprising creating an opening in a wall of the first blood vessel, wherein the distal portion is extended through the opening.

Example 32: The method of any example herein, in particular example 17, further comprising extending the distal portion into a branching blood vessel.

Example 33: The method of any example herein, in particular example 32, further comprising cutting the branching blood vessel to expose the distal portion.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method comprising:
percutaneously delivering an implant into a first blood vessel, the implant having a flexible and elastic structure;
extending at least a distal portion of the implant out of the first blood vessel while retaining at least a proximal portion of the implant within the first blood vessel; and
injecting the implant with a fluid, the fluid being freely moveable between the proximal portion and the distal portion, the proximal portion being shape-set to form a bulb.

2. The method of claim 1, wherein the implant is shape-set to form an approximately 90-degree bend to offset the proximal portion approximately 90-degrees relative to the distal portion.

3. The method of claim 1, wherein the implant comprises a wire frame and a covering.

4. The method of claim 3, wherein the wire frame is at least partially composed of a shape-memory alloy.

5. The method of claim 1, wherein the implant is shape-set to form a bulb at the distal portion.

6. The method of claim 1, further comprising percutaneously delivering a stent into the first blood vessel and adjacent to the proximal portion of the implant to press fluid out of the proximal portion of the implant and into the distal portion of the implant in response to increased blood pressure through the first blood vessel.

7. The method of claim 6, wherein the implant is coupled to the stent.

8. The method of claim 6, wherein the implant is disposed between the stent and a wall of the first blood vessel.

9. The method of claim 6, wherein the stent comprises a wire frame and a covering.

10. The method of claim 6, wherein the implant has a smaller diameter than the stent.

11. The method of claim 1, further comprising creating an opening in a wall of the first blood vessel, wherein the distal portion is extended through the opening.

12. The method of claim 1, further comprising extending the distal portion into a branching blood vessel.

13. The method of claim 12, further comprising cutting the branching blood vessel to expose the distal portion.

14. The method of claim 1, wherein the proximal portion is shape-set to expand and the distal portion is shape-set to not expand in response to injection of fluid into the implant.

15. A method comprising:
percutaneously delivering an implant into a first blood vessel, the implant having a flexible and elastic structure;
extending at least a distal portion of the implant out of the first blood vessel while retaining at least a proximal portion of the implant within the first blood vessel;
injecting the implant with a fluid, the fluid being freely moveable between the proximal portion and the distal portion; and
percutaneously delivering a stent into the first blood vessel and adjacent to the proximal portion of the implant to press fluid out of the proximal portion of the implant and into the distal portion of the implant in response to increased blood pressure through the first blood vessel.

16. The method of claim 15, wherein the proximal portion is shape-set to form a bulb.

17. The method of claim 15, further comprising creating an opening in a wall of the first blood vessel, wherein the distal portion is extended through the opening.

18. A method comprising:
percutaneously delivering an implant into a first blood vessel, the implant having a flexible and elastic structure;
creating an opening in a wall of the first blood vessel;
extending at least a distal portion of the implant through the opening and out of the first blood vessel while retaining at least a proximal portion of the implant within the first blood vessel; and
injecting the implant with a fluid, the fluid being freely moveable between the proximal portion and the distal portion.

19. The method of claim 18, further comprising extending the distal portion into a branching blood vessel.

20. The method of claim 19, further comprising cutting the branching blood vessel to expose the distal portion.
